(19) United States
(12) Patent Application Publication
(10) Pub. No.: US 2020/0363299 A1
(43) Pub. Date: Nov. 19, 2020

(54) APPARATUS FOR AUTOMATING PRETREATMENT OF NUCLEIC ACID DETECTION

(71) Applicant: Hangzhou Gene-Meta Medical Device Co., Ltd., Hangzhou (CN)

(72) Inventors: Baochun Ye, Hangzhou (CN); Fei Huang, Hangzhou (CN); Yunying Zhu, Hangzhou (CN)

(73) Assignee: Hangzhou Gene-Meta Medical Device Co., Ltd., Hangzhou (CN)

(21) Appl. No.: 16/753,786

(22) PCT Filed: May 17, 2017
(86) PCT No.: PCT/CN2017/084700

§ 371 Date: Apr. 5, 2020

(30) Foreign Application Priority Data
Nov. 23, 2016 (CN) 2016110464675
Dec. 14, 2016 (CN) 2016111513912

(51) Int. Cl. G01N 1/34; B01L 3/00; C12M 3/04; B01L 7/00; C12Q 1/6806

(52) U.S. Cl. CPC G01N 1/34; B01L 3/502792; B01L 2300/069; B01L 7/52; C12Q 1/6806; C12M 27/10

(57) ABSTRACT

An automated apparatus for pre-processing of nucleic acid detection includes a reagent plate to place sample and pre-processing reagent; a base for placing the reagent plate provided with a temperature control module and a blending module; a magnetic separation module for magnetic particles separation, cleaning and elution; a pipetting device for transferring the reagent in the reagent plate comprising a pipetting module, a rotation module and a pipetting tip; a mobile rail platform for supporting the pipetting device; and a host computer for controlling the temperature control module, rotation module, blending module, magnetic separation module and pipetting module. The automated apparatus for pre-processing of nucleic acid detection of the present invention can execute full automation of pre-processing of nucleic acid detection including extraction, library construction and product purification. The final product can be directly used in the unattended operation of high-throughput sequencing.

### FIELD OF THE INVENTION

The present invention relates to nucleic acid molecular detection, and more specially relates to an automated apparatus for pre-processing of nucleic acid detection. The present invention can realize full automation of pre-processing of nucleic acid detection including nucleic acid extraction, amplification, purification, etc. The post-processing sample can be detected by an external matching sequencer. The present invention can simplify detection process, reduce the chances of error and improves the reliability of the whole detection.

### BACKGROUND

The technology of high-throughput sequencing is a new molecular detection technology that has been developed in the last decade. The prevailing high-throughput sequencers available in the market are mainly provided by Illumina, ThermoFisher, etc. Before detection, any high-throughput sequencing platform requires the extraction of DNA or RNA from biological sample and the construction of sequencing library of the DNA sample to be tested by connecting the adaptor DNA sequences commonly used by the sequencer to both ends of the test DNA. Because many tool enzymes and buffer systems have been added during the library construction process, which are not conducive to later reactions, further DNA purification operation is needed to remove various enzymes and buffers such that the test fragment DNA can have sequencing reactions at different chips of the sequencer.

Conventional pre-processing of high-throughput sequencing contains three steps: nucleic acid extraction, library construction, and purification.

Nucleic acid extraction: nucleic acid detection firstly requires nucleic acid extraction from the sample. Common methods contain ethanol precipitation method, silica gel column binding method, glass beads method, and magnetic particles binding separation method.

The basic principle of all methods is four steps: lysing the sample—combining—washing—eluting. The entire course for a single sample takes about 40 minutes.

Library construction: The basic concept of library construction is to add DNA adaptors that matches with the subsequent sequencing equipment to the DNA to be tested. The basic workflow is as follows: 1. end-filling; 2. add an A at the end of the nucleotide; 3. link DNA adaptors; 4. PCR amplification. Because individual enzyme and buffer system are needed in each step, DNA purification based on silica gel column or magnetic particles is also needed in each reaction during the course of a normal library construction of high-throughput sequencing. Namely, normal workflow of library construction of high-throughput sequencing has seven steps: 1. end-filling; 2. purification; 3. add an A at the end of the nucleotide; 4. purification; 5. link DNA adaptor; 6. purification; 7. PCR amplification.

Purification: various tool enzymes are added in the course of library construction. To purify the sample, DNA solution is combined with media (such as magnetic particles, silicone membrane or glass beads), washed twice with a cleaning solution to remove various impurities, and finally eluted DNA with an eluant.

Thus, all steps from a primary sample to a final sequencing library have been completed. The whole workflow needs 5-8 hours and labeling test tubes is needed at each step to avoid making mistaking with the samples. The whole workflow is time-consuming and laborious.

Aiming at avoiding the above-mentioned complicated steps, some devices are available on the market to replace manual operations. The high-throughput sequencing is a brand-new technology on the market. There is a single apparatus for each step, such as various nucleic acid attractors for nucleic acid extraction, automated pipetting workstations for library construction, and manual or automated pipetting workstations for library purification. A nucleic acid extractor can perform 12-96 well nucleic acid extraction. An operator places a sample in the extractor, takes out the attracted sample after 40 minutes, and labels it for use in subsequent tests.

For pre-processing of nucleic acid, the steps of reaction setup, temperature control, and nixing can be executed with a pipetting workstation. Before operations, the operator places the necessary corollary reagents on the worktable of the workstation, sets up program, labels the positions of input test tubes and output test tubes, and then can start the operations. Because the pipetting workstation is an open platform and the operator must periodically seal and open the membrane of the 96-well plate or periodically open and close tube covers during the multi-step test operation. Though part of work may be done with the workstation, yet a lot of manual intervention is needed and unattended operation throughout the whole process is impossible. Many steps need manual labeling, sequencing and setting up. Furthermore, once the test is started, the workflow cannot be temporarily added or changed. Temporary handling of sample or changing test workflow will affect the samples in the whole test.

### SUMMARY OF THE INVENTION

The present invention aims at overcoming the deficiency in prior art by providing an automated apparatus for pre-processing of nucleic acid detection that can execute full automation of pre-processing of nucleic acid detection including extraction, library construction and product purification. The final product can be directly used in high-throughput sequencing without user intervention.

An automated apparatus for pre-processing of nucleic acid detection, comprising:

A reagent plate for placing samples and pre-processing reagents;

A base for the placing reagent plate provided with a temperature control module and a blending module;

A magnetic separation module for separation, cleaning and elution of magnetic particles;

A pipetting device for transferring the reagent in the reagent plate comprising a pipetting module and a pipetting tip;

A mobile rail platform for supporting the pipetting device; and

A host computer for controlling the temperature control module, rotation module, blending module, magnetic separation module and pipetting module.

The present invention also provides another automated apparatus for pre-processing of nucleic acid detection, comprising:

A reagent plate for placing samples and pre-processing reagents;

A base for placing the reagent plate provided with a temperature control module and a rotation module;

A magnetic separation module for magnetic particles separation, cleaning and elution;

A pipetting device for transferring the reagent in the reagent plate comprising a pipetting module and a pipetting tip; and

A host computer for controlling the temperature control module, rotation module, magnetic separation module and pipetting module.

The automated apparatus for pre-processing of nucleic acid detection of the present invention can automatically execute the processes of the extraction, library construction, and purification of nucleic acid. Each apparatus can perform independent closed operation as a unit or expand to unlimited number in series for parallel detection of multi-samples.

Furthermore, the aluminum foil sealing is provided with a pipe support and a silica gel layer between the pipe support and the cover. During pre-processing of nucleic acid detection, first remove the cover of the reagent plate. The pipetting device (such as pipetting tip) of the apparatus can directly puncture the silica gel and aluminum foil sealing and enter the cavity for pipetting. After the operation is completed, the pipetting device will exit the aluminum foil and silica gel. Through hole left in the aluminum foil sealing will be sealed by silica gel resilience, and the reagent in the reagent hole will not be volatilized and affect the whole test.

The shape of said reagent plate can be round, square or rectangular, or even irregular. Reagent holes can be optimally arranged in accordance with the shape of the reagent plate. Preferably, disposable individual reagent plate is adopted for cellular reagent plate. After the sample to be tested is placed in the reagent plate, all pre-process workflow of nucleic acid detection can be completed inside said reagent plate.

The pre-processing reagents for nucleic acid sequencing comprise nucleic acid attracting reagent, library construction reagent and DNA purification reagent, and include but not limited to the reagents in the steps of nucleic acid end-filling, add A, adaptor ligation, and purification.

Said reagent holes comprise lysis hole, magnetic beads hole, extraction hole, library construction hole, PCR reacting hole, purification hole, final sample hole and waste fluid hole.

Said base is provided with a rotation module. The reagent plate can move and rotate through the rotation module in the base.

Said pipetting module comprises a pipetting pump.

Said magnetic separation module comprises one or more mobile magnets installed in the base and adjacent to reagent plate.

The apparatus of the present invention comprises temperature control module, pipetting module, blending module and magnetic separation module for performing the functions of temperature control, pipetting, blending and magnetic separation. The blending module can generate horizontal vortex and vertical blending inside the apparatus. The temperature control module can execute the treatment of raising, low temperature and changing temperature in one or more steps. The pipetting module can execute fluid transfer among different reagent holes in the reagent plate by one or more pipetting tips included in the apparatus or the reagent plate. The magnetic separation module can execute the work of magnetic particle separation, cleaning and elution by installed electric magnet or permanent magnet.

In one embodiment, the automated apparatus for pre-processing of nucleic acid detection uses a disposable reagent plate which contains extracting reagent, library construction reagent and DNA purification reagent. After samples are added in the sample hole of the reagent plate, the apparatus will execute pipetting to add lysate; blend for 10 minutes by the apparatus; add binding buffer and magnetic beads with pipetting tip and blend for 10 minutes; absorb magnetic beads with magnetic separation module, remove supernatant with pipette, add 500 ul cleaning fluid, blend magnetic beads for 2 minutes, and remove cleaning fluid with magnetic separation module, and repeat for three times; add 30 ul eluant and blend it with magnetic beads, heat at 60° C. for 5 minutes; transfer supernatant to library construction hole with the pipette; add end-filling enzyme with pipetting tip, let stand 37° C. for 30 minutes and inactivate at 75° C. for 20 minutes; add DNA adaptor and DNA ligase with pipetting tip, let stand 20° C. for 30 minutes and inactivate at 75° C. for 20 minutes; transfer the solution to purification hole with the pipette, add magnetic beads and purification binding buffer, and blend for 10 minutes; absorb magnetic beads with magnetic separation module to remove supernatant, and clean magnetic beads twice with 500 ul cleaning fluid; add 50 ul eluant in magnetic beads, heat at 60° C. for 5 minutes, transfer DNA to final sample cavity, thus finishing the pre-process of sample nucleic acid.

The present invention can realize full automated unattended pre-processing of nucleic acid detection in a closed independent reagent plate. An operator can place a sample in the reagent plate and allow the apparatus to run automatically. The product can be directly used for later sequencing analysis. The present invention can significantly simplify the testing workflow, avoid complicated normal manual operations, and also avoid links such as frequently labeling test tubes and manual pipetting when operate together with nucleic acid extraction device and pipetting workstation, so that eradicate the possibility of sample confusion on the basis of testing workflow.

The automated apparatus for pre-processing of nucleic acid detection of the present invention has following advantages:
(1) Full automation: The apparatus fully automatically execute the pre-processing steps of nucleic acid detection including nucleic acid extraction, end-filling, adding A, adaptor connection and product purification. Manual operation does not exceed 5 minutes in the whole course. An operator just needs to place the sample in the reagent plate and put the reagent plate in the apparatus, and the consequent workflow will be fully automatically executed without user intervention.
(2) Closed operation. The reagent plate goes in a closed system, guaranteeing samples not confused and no mutual contamination.
(3) Quickness. The direct links between different testing steps realized by the apparatus can reduce the sample labeling work needed in many links, thus greatly accelerating the whole test.
(4) Programmability. The apparatus has modules of blending, pipetting, temperature control and magnetic separation, enabling re-development on the apparatus according to actual needs, having high scalability.
(5) High throughput. Based on one-sample-one-operating-unit, the apparatus can be exaggerated unlimitedly by connecting units in series, thus realizing high throughput flexibly.

### BRIEF DESCRIPTION OF THE DRAWINGS

FIG. 1 is a structure diagram of the automated apparatus for pre-processing of nucleic acid detection.

FIG. 2 is another structure diagram of the automated apparatus for pre-processing of nucleic acid detection.

FIG. 3 is a schematic view of a reagent plate.

FIG. 4 is a partial sectional view of the reagent plate.

FIG. 5 is a schematic view of multiple automated apparatuses for pre-processing of nucleic acid detection in series arrangement to realize expansion.

### DETAILED DESCRIPTION

### EXAMPLE 1

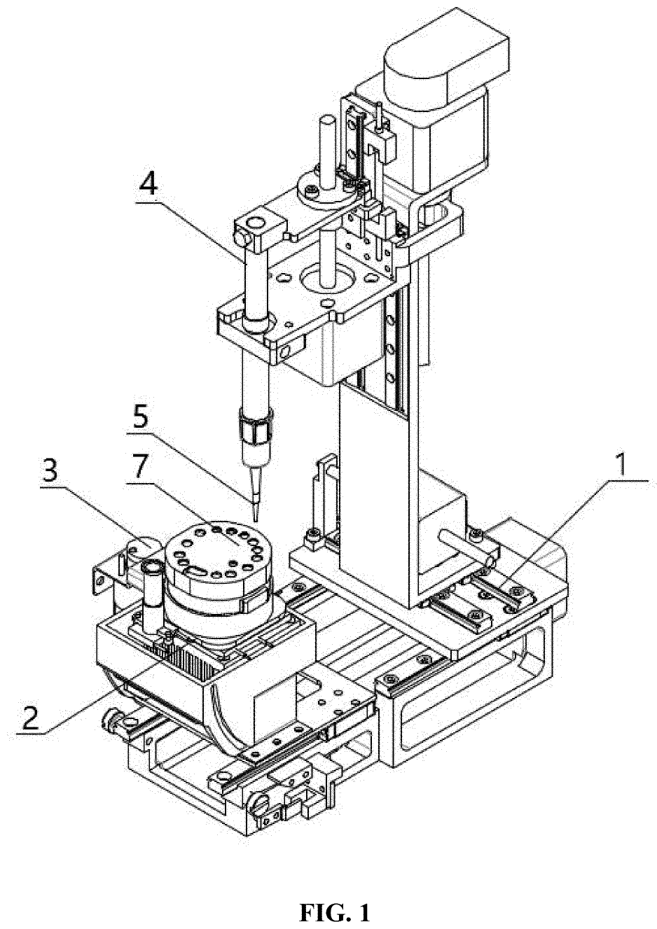

An automated apparatus for pre-processing of nucleic acid detection shown in FIG. 1 comprises a mobile rail platform 1, a base 2, one or more mobile magnets 3, a pipetting pump 4 and a pipetting tip 5. The pipetting tip 5 is provided on the pipetting pump 4 and the pipetting pump 4 can move along the mobile rail platform 1 and rotate through the rotation module. The mobile rail platform 1 can drive the pipetting pump and the pipetting tip to move and rotate above the reagent plate to realize pipetting. The position of the base 2 corresponding to the reagent plate is provided with a temperature control module. The base 2 fits the reagent plate to supply temperature control. The reagent plate can move and rotate through the rotation module in the base 2.

### EXAMPLE 2

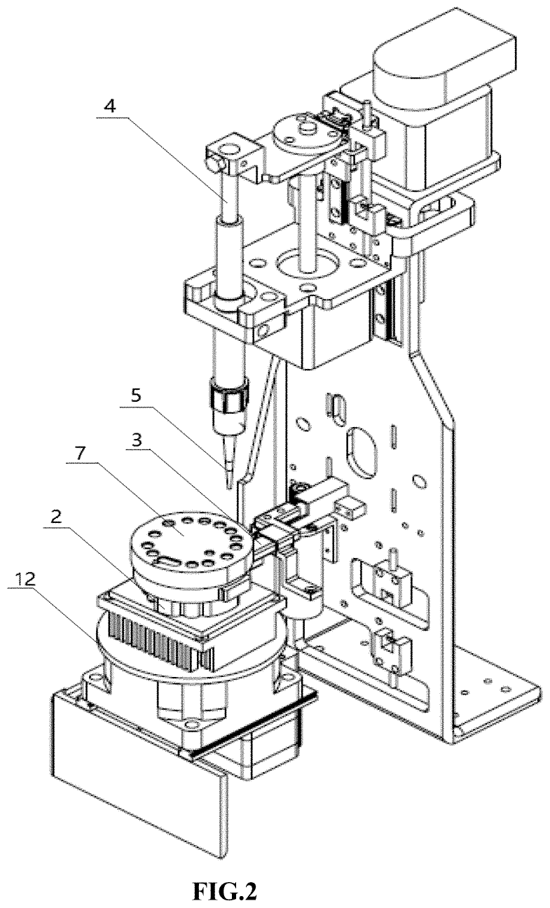

An automated apparatus for pre-processing of nucleic acid detection shown in FIG. 2 comprises a rotation device 12, a base 2, one or more mobile magnets 3, a pipetting pump 4 and a pipetting tip 5. The pipetting tip 5 is provided in the pipetting pump 4. The rotation device 12 is provided in the base 2 and the position of base 2 corresponding to the reagent plate is provided with a temperature control module. The rotation of the rotation device 12 can synchronously bring the rotation of the temperature control module. The base 2 fits the reagent plate and the temperature control module supplies temperature control. The reagent plate can move and rotate through the rotation device 12 in the base 2.

### EXAMPLE 3

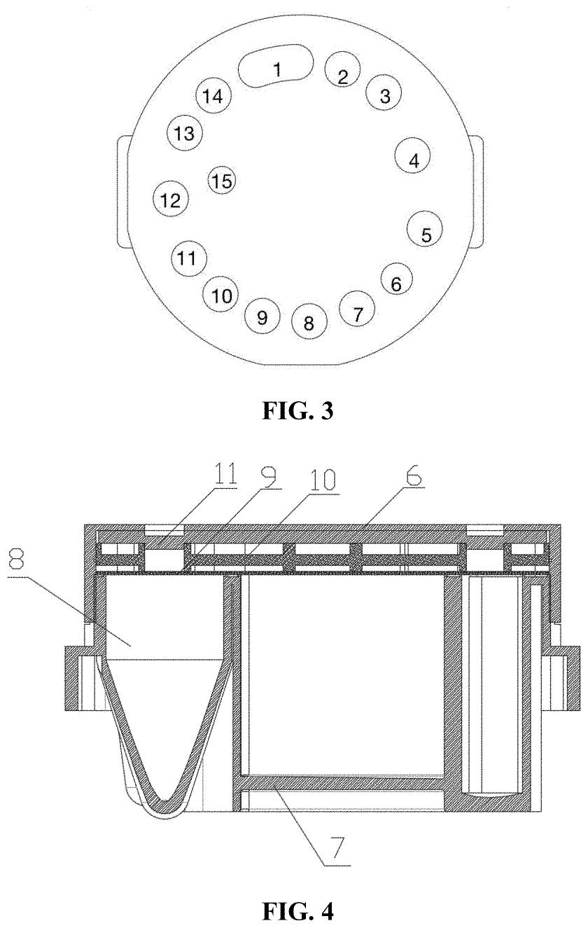

The reagent plate shown in FIG. 3-4 comprises a cover 6 and a reagent tray 7. The reagent tray 7 has several reagent holes 8. The reagent holes 8 contain separately the reagent for pre-processing of nucleic acid sequencing and are provided with aluminum foil sealing 9 at their opening mouths. The aluminum foil sealing 9 is provided with a pipe support 10, and a silica gel layer 11 is provided between the pipe support and the cover. The reagent holes comprise: (1)—lysis hole (6M guanidine hydrochloride, 20% isopropanol); (2)—magnetic beads hole (40 ul alkylated magnetic bead); (3)—cleaning solution (80% ethanol); (4)—binding solution (60% isopropanol); (5)—eluate (pure water pH8.0); (6)—extraction hole; (7)—library construction hole; (8)—end-filling enzyme mixing hole (Klenow DNA polymerase I, T4 polynucleotide kinase); (9)—adaptor ligation hybrid hole (T4 DNA ligase); (10)—purification hole (40 ul alkylated magnetic beads); (11)—reserved hole; (12)—final sample hole; (13)—waste liquid hole; (14)—reserved hole; (15)—reserved hole;

### EXAMPLE 4

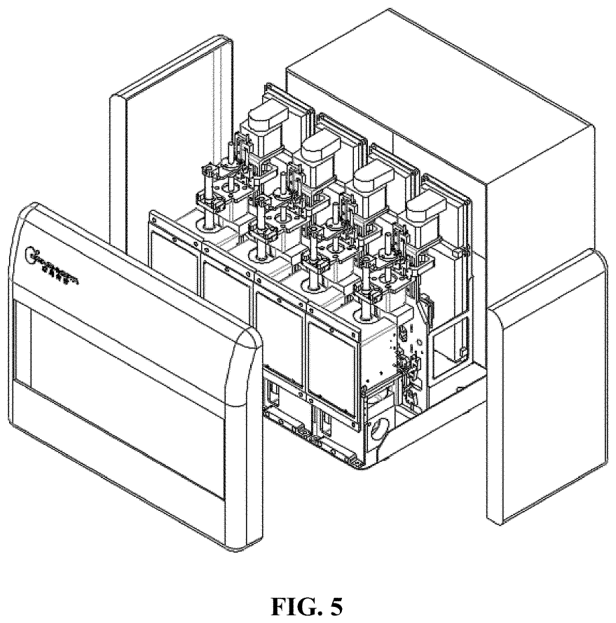

FIG. 5 is a schematic view of four automated apparatuses for pre-processing of nucleic acid detection in series arrangement according to Example 1 or Example 2. Multiple series arrangements can realize simultaneous detection of multiple samples as needed.

### EXAMPLE 5

The plasma free DNA is small-chunk fragmented DNA with fragment length of 100-300 BP and peak value of about 160 BP. In 1997, Prof. LU, Yuming found cell free fetal DNA in maternal peripheral plasma and created the method of analyzing whether there is chromosomal abnormality in the fetus of pregnant women by testing free DNA. Circulating tumor DNA (ct DNA) from tumor cells also exists in plasma and is widely used in liquid biopsy of tumor for noninvasive early diagnosis, accompanying diagnosis and prognosis follow-up and so on.

Normal testing method comprises three steps: first, extracting DNA from clinical sample including plasma, then pre-processing of nucleic acid, and finally detecting. Finishing the whole sample detection needs 6-13 hours, in which sample nucleic acid extraction and the pre-processing of nucleic acid detection are very tedious.

The automated apparatus for pre-processing of nucleic acid detection of the present invention can realize full automated unattended pre-processing of nucleic acid detection in a closed independent reagent plate. An operator can place a sample in the reagent plate and allow the apparatus to run automatically. The product can be directly used for later sequencing analysis. The specific operation method is as follows:

Place 600 ul plasma sample in the automated apparatus for pre-processing of nucleic acid detection for full automated high-throughput library construction.
1. Add 600 ul plasma in the lysis hole of the reagent plate which contains 1.2 ml plasma lysate, 6M guanidine hydrochloride and 20% isopropanol;
2. Blend the solution for 5 minutes with the blending module, and transfer 600 ul solution in sequence with the pipette to the extraction hole to combine with magnetic beads (40 ul silylated beads);
3. Conduct magnetic separation through the mobile magnets and transfer the supernatant to the waste liquid hole;
4. Clean the magnetic beads three times with 500 ul cleaning fluid (80% alcohol) and transfer the supernatant to the waste liquid hole;
5. Transfer 60 ul eluant to the extraction hole and elute DNA at 56° C. for 10 minutes;
6. Transfer DNA to the library construction hole and add 10 ul of end-filling enzyme (DNA polymerase I, T4 polynucleotide kinase) mixture;
7. Maintain at 37° C. for 30 minutes and then inactivate at 75° C. for 20 minutes;
8. Add 5 ul of DNA adaptor and 20 ul of DNA ligase (T4 nucleotide ligase) mixture;
9. Maintain at 16° C. for 20 minutes and then inactivate at 75° C. for 20 minutes;
10. Transfer all product to purification hole with pipette, and the purification hole contains the magnetic beads needed for purification reaction;
11. Blend sample and magnetic beads up and down with pipette and let stand for 5 minutes, place the mobile magnets and let stand for another 5 minutes, transfer the supernatant to the waste fluid hole with pipette;
12. Clean the magnetic beads twice with 500 ul cleaning fluid, adsorb the magnetic beads with the mobile magnets, and transfer the supernatant to the waste liquid hole;
13. Add 40 ul eluant into purification hole, elute DNA, and transfer the liquid to final sample hole with pipette;
14. The final product can be directly used for consequent high-throughput sequencing.

### EXAMPLE 6

The object studied by transcriptome sequencing is the total sum of all RNA transcribed in a certain cell under a certain function, mainly including mRNA and non-coding RNA. The transcriptome analysis is the basis and starting point of the study of gene function and structure, and by new-generation high throughput sequencing can fully and rapidly obtain nearly all transcript sequence data of a specific tissue or organ of a species under a circumstance. The transcriptome analysis has been widely applied in areas such as fundamental research, clinical diagnosis and drug development, etc.

Normal testing method comprises three steps: first, extracting RNA from clinical sample including whole blood and plasma, then pre-processing of nucleic acid, and finally detecting. Finishing the whole sample detection needs 6-30 hours. In which the sample nucleic acid extraction and pre-processing of nucleic acid detection are very complicated.

The automated apparatus for pre-processing of nucleic acid detection of the present invention can realize full automated unattended pre-processing of nucleic acid detection in a closed independent reagent plate. An operator can place a sample in the reagent plate and allow the apparatus to run automatically. The product can be directly used for the later sequencing analysis. The specific mode of operation is as follows:

Place 200 ul of whole blood sample in the automated apparatus for pre-processing of nucleic acid detection to realize full automated library construction of transcription.
1. First add 200 ul samples in the sample holes of the reagent plate, then add 300 ul lysate with pipetting tip;
2. Blend for 10 minutes with blending module;
3. Add 200 ul binding buffer and 10 ul magnetic beads with pipetting tip and blend for 10 minutes;
4. Adsorb magnetic beads with magnetic separation module, and remove supernatant with the pipette;
5. Add 500 ul cleaning fluid, blend magnetic beads for 2 minutes, and remove cleaning fluid with magnetic separation module, and repeat for three times;
6. Add 30 ul eluant and blend it with magnetic beads, heat at 60° C. for 5 minutes;
7. Transfer supernatant to extraction hole with the pipette;
8. Let stand in the extraction hole which contains 30 ul oligo dT magnetic beads at 65° C. for 30 minutes;
9. Remove supernatant with magnetic separation module, clean with 500 ul cleaning fluid and then remove supernatant with magnetic separation module again;
10. Add 30 ul eluant in extraction hole, let stand at 60° C. for 5 minutes, and transfer supernatant cDNA to library construction hole with the pipetting device;
11. Add end-filling enzyme and dATP with pipetting tip, let stand 37° C. for 30 minutes and inactivate at 65° C. for 5 minutes;
12. Add DNA adaptor and ligase with pipetting tip, let stand 20° C. for 30 minutes and inactivate at 65° C. for 5 minutes;
13. Add 10 ul magnetic beads and 200 ul purification binding buffer, and blend for 10 minutes;
14. Adsorb magnetic beads with magnetic separation module to remove supernatant, and clean magnetic beads twice with 500 ul cleaning fluid;
15. Add 50 ul eluant in magnetic beads, and heat at 60° C. for 5 minutes;
16. Transfer DNA to final sample hole with the pipette, ending the test with the automated processing apparatus;
17. The final DNA can be directly used for consequent high-throughput sequencing.

What claims is:

1. An automated apparatus for pre-processing of nucleic acid detection, comprising:
a reagent plate for placing samples and pre-processing reagent;
a base for placing said reagent plate provided with a temperature control module and a blending module;
a magnetic separation module for separation, cleaning and elution of magnetic particles;
a pipetting device for transferring the reagents in the reagent plate comprising a pipetting module, a rotation module and a pipetting tip;
a mobile rail platform for supporting the pipetting device; and
a host computer for controlling the temperature control module, rotation module, blending module, magnetic separation module and pipetting module.

2. The automated apparatus for pre-processing of nucleic acid detection as defined in claim 1, wherein said reagent plate comprises a cover and a reagent tray with several reagent holes containing separately the pre-processing reagents for nucleic acid sequencing and provided with aluminum foil sealing at their openings.

3. The automated apparatus for pre-processing of nucleic acid detection as defined in claim 1, wherein said aluminum foil sealing is provided with a pipe support and a silica gel layer between the pipe support and the cover.

4. The automated apparatus for pre-processing of nucleic acid detection as defined in claim 1, wherein the shape of said reagent plate can be round, square or rectangular.

5. The automated apparatus for pre-processing of nucleic acid detection as defined in claim 1, wherein said pre-processing reagents for nucleic acid sequencing comprise nucleic acid extracting reagent, library construction reagent and DNA purification reagent.

6. The automated apparatus for pre-processing of nucleic acid detection as defined in claim 1, wherein reagent holes comprise lysis hole, magnetic beads hole, extraction hole, library construction hole, PCR reacting hole, purification hole, final sample hole and waste fluid hole.

7. The automated apparatus for pre-processing of nucleic acid detection as defined in claim 1, wherein said base is provided with a rotation module.

8. The automated apparatus for pre-processing of nucleic acid detection as defined in claim 1, wherein said pipetting module comprises a pipetting pump.

9. The automated apparatus for pre-processing of nucleic acid detection as defined in claim 1, wherein said magnetic separation module comprises one or more mobile magnets installed in the base and adjacent to said reagent plate.

10. An automated apparatus for pre-processing of nucleic acid detection, comprising:
a reagent plate for placing samples and pre-processing reagent;
a base for placing said reagent plate provided with a temperature control module and a rotation module;
a magnetic separation module for separation, cleaning and elution of magnetic particles;
a pipetting device for transferring the reagent in the reagent plate comprising a pipetting module and a pipetting tip; and
a host computer for controlling the temperature control module, rotation module, magnetic separation module and pipetting module.

11. The automated apparatus for pre-processing of nucleic acid detection as defined in claim 10, wherein said reagent plate comprises a cover and a reagent tray with several reagent holes containing separately the pre-processing reagents for nucleic acid sequencing and provided with aluminum foil sealing at their openings.

12. The automated apparatus for pre-processing of nucleic acid detection as defined in claim 10, wherein said aluminum foil sealing is provided with a pipe support and a silica gel layer between the pipe support and the cover.

13. The automated apparatus for pre-processing of nucleic acid detection as defined in claim 10, wherein the shape of said reagent plate is round.

14. The automated apparatus for pre-processing of nucleic acid detection as defined in claim 10, wherein said pre-processing reagents for nucleic acid sequencing comprise nucleic acid extracting reagent, library construction reagent and DNA purification reagent.

15. The automated apparatus for pre-processing of nucleic acid detection as defined in claim 10, wherein reagent holes comprise lysis hole, magnetic beads hole, extraction hole, library construction hole, PCR reacting hole, purification hole, final sample hole and waste fluid hole.

16. The automated apparatus for pre-processing of nucleic acid detection as defined in claim 10, wherein the rotation of said rotation module in said base brings the rotation of temperature module synchronously.

17. The automated apparatus for pre-processing of nucleic acid detection as defined in claim 10, wherein said pipetting module comprises a pipetting pump.

18. The automated apparatus for pre-processing of nucleic acid detection as defined in claim 10, wherein said magnetic separation module comprises one or more mobile magnets installed in said base and adjacent to said reagent plate.
